(19) United States
(12) Patent Application Publication
(10) Pub. No.: US 2010/0145362 A1
(43) Pub. Date: Jun. 10, 2010

(54) APPARATUS AND METHODS FOR CONTROLLED RELEASE OF TACKING DEVICES

(75) Inventor: Tyler E. McLawhorn, Winston-Salem, NC (US)

Correspondence Address: BRINKS HOFER GILSON & LIONE/CHICAGO/COOK, PO BOX 10395, CHICAGO, IL, 60610, US

(73) Assignee: Wilson-Cook Medical Inc., Winston-Salem, NC (US)

(21) Appl. No.: 12/632,281

(22) Filed: Dec. 7, 2009

Related U.S. Application Data
(60) Provisional application No. 61/120,962, filed on Dec. 9, 2008.

(51) Int. Cl. A61B 17/10; A61B 17/04

(52) U.S. Cl. 606/139; 606/232

(57) ABSTRACT

The present embodiments provide a tacking device for engaging tissue, which may be useful for facilitating closure of a bodily opening. The tacking device comprises a main body having proximal and distal ends, and at least one distal deployable member having contracted and expanded states that extends distally from the distal end of the main body. In use, after the distal deployable members have been at least partially expanded at a preliminary location, the distal deployable members may be contracted to permit repositioning at a different, final location. In one exemplary method, at least two tacking devices may be deployed to at least partially surround an opening in tissue, and a suture coupled to the first and second tacking devices may be actuated to facilitate closure of the opening.

### PRIORITY CLAIM

This invention claims the benefit of priority of U.S. Provisional Application Ser. No. 61/120,962, entitled “Apparatus and Methods for Controlled Release of Tacking Devices,” filed Dec. 9, 2008, the disclosure of which is hereby incorporated by reference in its entirety.

### BACKGROUND

The present embodiments relate generally to medical devices, and more particularly, to devices for engaging tissue or facilitating closure of a bodily opening.

Perforations in tissue or bodily walls may be formed intentionally or unintentionally. For example, an unintentional ventral abdominal hernia may be formed in the abdominal wall due to heavy lifting, coughing, strain imposed during a bowel movement or urination, fluid in the abdominal cavity, or other reasons.

Intentional perforations may be formed, for example, during surgical procedures such as translumenal procedures. In a translumenal procedure, one or more instruments, such as an endoscope, may be inserted through a visceral wall, such as the stomach wall. During a translumenal procedure, a closure instrument may be used to close the perforation in the visceral wall. Depending on the structure comprising the perforation, it may be difficult to adequately close the perforation and prevent leakage of bodily fluids.

Attempts to seal perforations have been performed by coupling a graft member to tissue. For example, a graft material such as a mesh or patch may be disposed to overlap with tissue surrounding the perforation. The graft material then may be secured to the surrounding tissue in an attempt to effectively cover and seal the perforation. In order to secure the graft material to the surrounding tissue, sutures commonly are manually threaded through the full thickness of the surrounding tissue, then tied down and knotted. However, such manual suturing techniques may be time consuming and/or difficult to perform. Moreover, when closing intentional openings formed during translumenal procedures, suturing techniques may permit leakage of bodily fluids, and may be unreliable and difficult to reproduce.

Further attempts to seal intentional openings in tissue have been performed using mechanical devices such as clips, tacks, staples, and fasteners. Such devices may be delivered towards a target tissue site and deployed to engage tissue surrounding the opening. However, typically once such mechanical devices are deployed, they are permanently engaged to the tissue and cannot be recaptured or repositioned, resulting in possible permanent deployment of such devices at an undesirable location.

### SUMMARY

The present embodiments provide a tacking device for engaging tissue, which may be useful for facilitating closure of a bodily opening. One embodiment of a tacking device comprises a main body having proximal and distal ends, and at least one distal deployable member having contracted and expanded states that extends distally from the distal end of the main body. In use, after the distal deployable members have been at least partially expanded at a preliminary location, the distal deployable members may be contracted to permit repositioning at a different, final location.

A first retainer may be disposed at the proximal end of the main body. The first retainer may be coupled to a second retainer prior to deployment of the tacking device, and may be configured to be disengaged from the second retainer after the tacking device is deployed at the final location. The tacking device preferably is delivered within a hollow lumen an insertion tool with the distal deployable members in the contracted state. The first and second retainers are securely coupled together when the insertion tool is positioned over both the first and second retainers, and the first retainer is configured to be disengaged from the second retainer when no longer covered by the insertion tool. After the distal deployable members have been deployed at the preliminary location, proximal retraction of an actuating wire coupled to the second retainer causes a corresponding proximal retraction of the first retainer, which in turn proximally retracts the distal deployable members within the insertion tool. The distal deployable members may assume the contracted state within the insertion tool, thereby facilitating repositioning of the insertion tool and subsequent deployment of the tacking device at the final location.

Advantageously, a tacking device provided in accordance with the present embodiments is recapturable after at least partial deployment of the distal deployable members. A physician therefore may reposition the tacking device if the preliminary deployment location or orientation within the tissue is undesirable, so long as the first and second retainers remain joined.

In one exemplary method, at least first and second tacking devices may be used for facilitating closure of a bodily opening in tissue. The first and second tacking devices may be deployed to at least partially surround the opening. A loop member of the first and second tacking devices is configured to receive a suture. In use, the first and second tacking members may be deployed, and the suture may be threaded through the loop members and actuated in a purse-string fashion to facilitate closure of the opening.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is a side view of a tacking device.

FIG. 2 is a side-sectional view of a distal region of an insertion tool and the tacking device of FIG. 1.

FIG. 3 is a top view of the distal region of the insertion tool and the tacking device of FIG. 2.

FIG. 4 is a side-sectional view of the distal region of an insertion device housing the insertion tool and the tacking device of FIGS. 1-3.

FIGS. 5-10 illustrate one exemplary method of use of the tacking device of FIGS. 1-4, with tissue shown from a side-sectional view, and device components shown from a side view for illustrative purposes.

### DETAILED DESCRIPTION OF THE PREFERRED EMBODIMENTS

In the present application, the term “proximal” refers to a direction that is generally towards a physician during a medical procedure, while the term “distal” refers to a direction that is generally towards a target site within a patient's anatomy during a medical procedure.

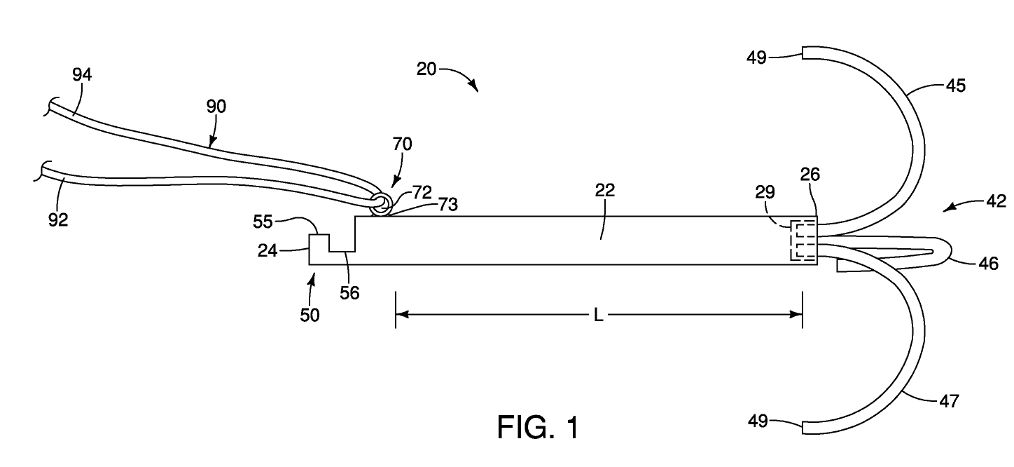

Referring now to FIG. 1, a first embodiment of a tacking device 20 is shown. In this embodiment, the tacking device 20 comprises a main body 22 having a proximal end 24 and a distal end 26. The tacking device 20 further comprises a distal deployment mechanism 42, which comprises three distal deployable members 45-47. The distal deployable members 45-47 extend distally from the distal end 26 of the main body 22, as shown in FIG. 1. The distal deployable members 45-47 each may be integrally formed with the main body 22 or formed separately and coupled to the main body 22. In the latter embodiment, a recess 29 may be formed in the distal end 26 of the main body 22, and proximal regions of the three distal deployable members 45-47 may be secured within the recess 29 of the main body 22 using an adhesive, frictional fit, mechanical device or other suitable mechanism. Alternatively, the recess 29 may be omitted and the distal deployable members 45-47 may be coupled or adhered to an exterior surface of the main body 22 near the distal end 26.

While three total distal deployable members 45-47 are depicted, it will be apparent that greater or fewer deployable members may be employed. Moreover, the distal deployable members 45-47 may comprise any shape suitable for engaging, penetrating and/or abutting tissue, for purposes explained further below, and need not necessarily assume the expanded shape depicted in FIG. 1.

The main body 22 may comprise any suitable shape and material. Solely by way of example, the main body 22 may comprise stainless steel or a biocompatible plastic. The main body 22 may be cylindrically-shaped, as depicted in FIG. 1, which may facilitate insertion through a lumen of an insertion tool 80, as explained below.

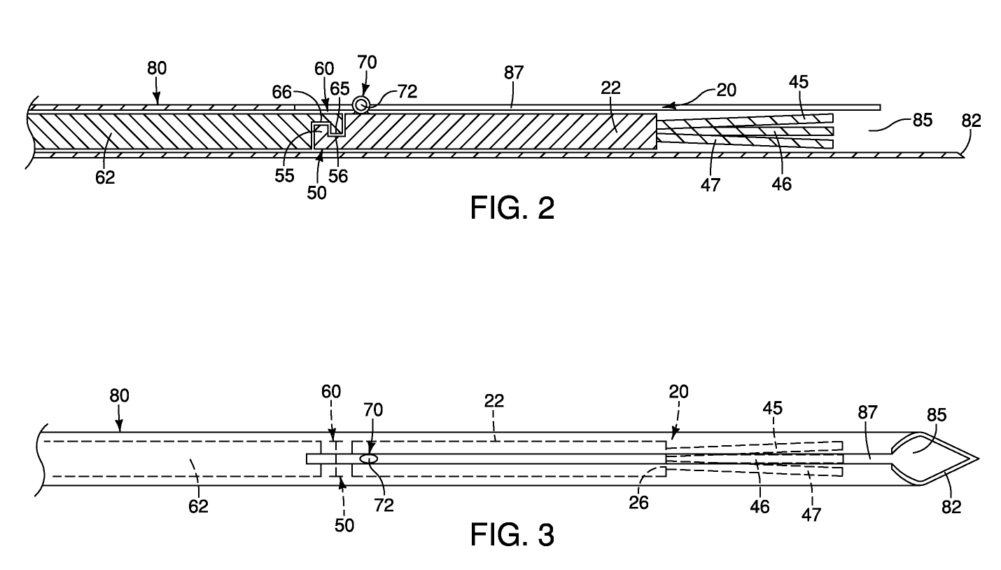

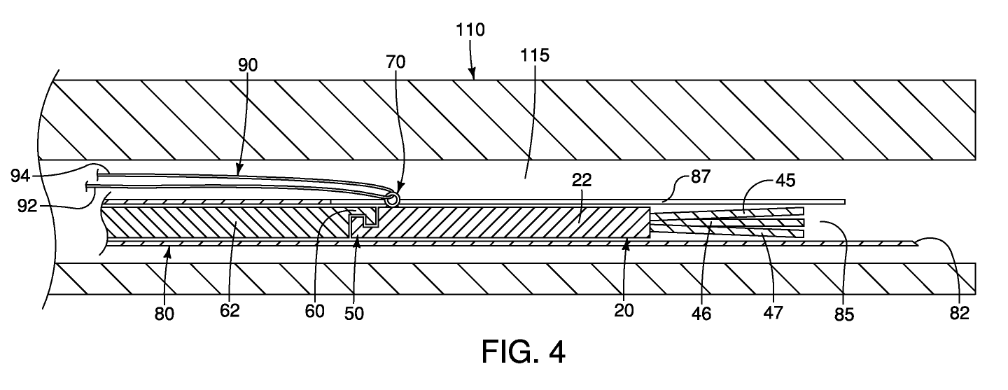

The distal deployable members 45-47 each comprise a contracted delivery configuration, as shown in FIGS. 2-3, and further comprise an expanded deployed configuration, as shown in FIG. 1. In one embodiment, each of the distal deployable members 45-47 comprises a hook-shaped configuration in the expanded state. For example, the distal deployable members 45-47 may comprise a curvature of about 90 to about 360 degrees in the expanded state, and more preferably about 180 degrees, as shown in FIG. 1. Where the distal deployable members 45-47 “retroflex” and comprises a curvature of about 180 degrees, the end regions 49 of the distal deployable members 45-47 are oriented substantially parallel to the main body 22. Moreover, the end regions 49 may be radially spaced apart from one another in the expanded state, as shown in FIG. 1. In this configuration, the end regions 49 may be well-suited for engaging, grasping, piercing and/or abutting tissue. In the embodiments depicted herein, the end regions 49 comprise blunt tips, but alternatively may comprise sharpened tips to facilitate piercing of tissue.

The distal deployable members 45-47 may comprise a shape-memory material, such as a nickel-titanium alloy (nitinol). If a shape-memory material such as nitinol is employed, the distal deployable members 45-47 may be manufactured such that they can assume the preconfigured expanded state shown in FIG. 1 upon application of a certain cold or hot medium. More specifically, a shape-memory material may undergo a substantially reversible phase transformation that allows it to “remember” and return to a previous shape or configuration. For example, in the case of nitinol, a transformation between an austenitic phase and a martensitic phase may occur by cooling and/or heating (shape memory effect) or by isothermally applying and/or removing stress (superelastic effect). Austenite is characteristically the stronger phase and martensite is the more easily deformable phase.

In an example of the shape-memory effect, a nickel-titanium alloy having an initial configuration in the austenitic phase may be cooled below a transformation temperature (M_{f}) to the martensitic phase and then deformed to a second configuration. Upon heating to another transformation temperature (A_{f}), the material may spontaneously return to its initial, predetermined configuration, as shown in FIG. 1. Generally, the memory effect is one-way, which means that the spontaneous change from one configuration to another occurs only upon heating. However, it is possible to obtain a two-way shape memory effect, in which a shape memory material spontaneously changes shape upon cooling as well as upon heating.

Alternatively, the distal deployable members 45-47 may be made from other metals and alloys that are biased, such that they may be restrained by the insertion tool 80 prior to deployment, but are inclined to return to their relaxed, expanded configuration upon deployment. Solely by way of example, the distal deployable members 45-47 may comprise other materials such as stainless steel, cobalt-chrome alloys, amorphous metals, tantalum, platinum, gold and titanium. The distal deployable members 45-47 also may be made from non-metallic materials, such as thermoplastics and other polymers. As noted above, the distal deployable members 45-47 may comprise any shape suitable for engaging, penetrating and/or abutting tissue, for purposes explained further below, and need not necessarily assume the curved shape depicted in FIG. 1.

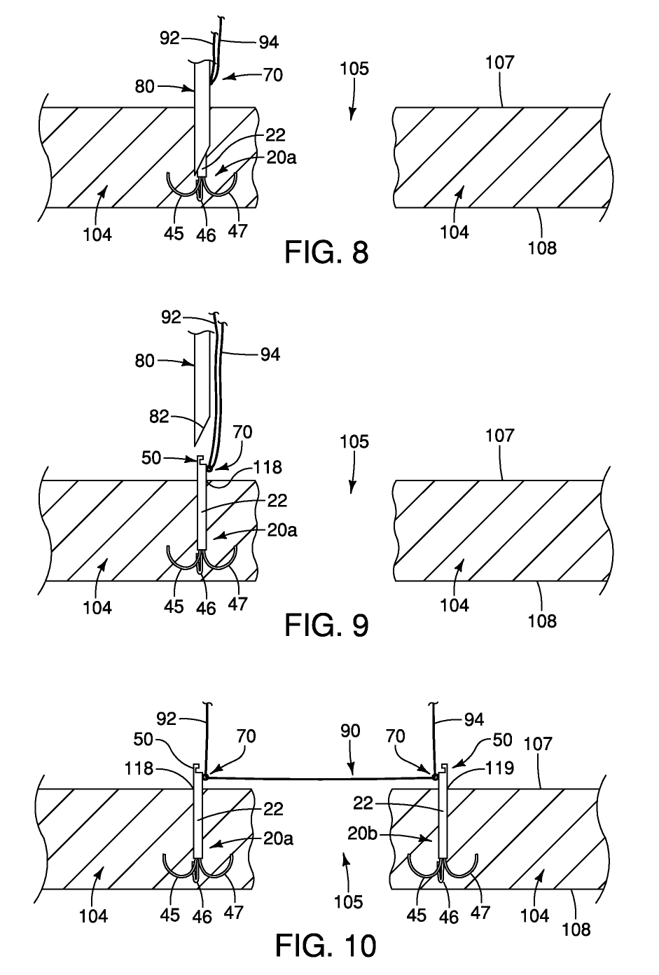

Referring still to FIG. 1, the tacking device 20 further comprises a first retainer 50 disposed at the proximal end 24 of the main body 22. As explained further below, the first retainer 50 may be used in conjunction with a second retainer 60 that may be advanced or retracted by a physician. In use, the first retainer 50 is configured to be proximally retracted, via the second retainer 60, after the distal deployable members 45-47 have been at least partially deployed at a preliminary location within tissue, thereby allowing for a controlled release of the tacking device 20 and repositioning of the distal deployable members 45-47 at a final location, as explained further in FIGS. 5-10 below.

Still further, the tacking device 20 comprises a loop member 70, which preferably is disposed near the proximal end 24 of the main body 22, for example, at a location just distal to the first retainer 50. The loop member 70 may be formed integrally with the main body 22, or alternatively, may be secured to the main body 22 using any suitable technique, such as a solder or weld 73. The loop member 70 comprises an aperture 72, which is sized to receive a suture 90 having first and second ends 92 and 94, as shown in FIG. 1. In use, the suture 90 is disposed through the aperture 72 of the loop member 70 and may be actuated to correspondingly actuate a portion of the tacking device 20. For example, as described further with respect to FIG. 10 below, the suture 90 may be actuated in a purse-string fashion to facilitate closure of an opening in tissue when at least two tacking devices are deployed to at least partially surround an opening in the tissue.

The dimension of the tacking device 20 may be tailored based on a particular surgical procedure, a particular patient's anatomy and/or other factors. However, for illustrative purposes, in a visceral wall closure operation as explained in FIGS. 5-10 below, the longitudinal length of the main body 22 may range from about 2 mm to about 15 mm, the straightened (delivery or non-curved) length of the distal deployable members 45-47 may range from about 5 mm to about 50 mm, the outer diameter of the main body 22 may range from about 0.3 mm to about 2.0 mm, and the outer diameter of the distal deployable member 45-47 may range from about 0.1 mm to about 1.0 mm. Further, a longitudinal distance L between the end regions 49 of the tacking device 20 and the loop member 70 may be selected to cause the loop member 70 to be positioned proximal to a mucosal layer of tissue when the distal deployable members 45-47 are engaged with the tissue, as depicted in FIGS. 9-10 below, and may range from about 2 mm to about 12 mm. Such dimensions are provided for reference purposes only and are not intended to be limiting.

Referring now to FIGS. 2-3, one or more tacking devices 20 may be delivered to a target site in a patient's anatomy using an insertion tool 80. In FIGS. 2-3, the tacking device 20 is shown in the contracted state, whereby the distal deployable members 45-47 may comprise a substantially longitudinally-oriented profile, i.e., oriented along a longitudinal axis of the insertion tool 80.

The insertion tool 80 may comprise a needle-like body having a sharpened distal tip 82 and a hollow lumen 85. If the insertion tool comprises a needle-like body, it may be manufactured from stainless steel or any other suitable material, and may comprise an endoscopic ultrasound (EUS), or echogenic, needle. Solely by way of example, a needle-like insertion tool may comprise the EchoTip® Ultrasound Needle, or the EchoTip® Ultra Endoscopic Ultrasound Needle, both manufactured by Cook Endoscopy of Winston-Salem, N.C. Further details of a suitable insertion tool comprising a needle-like body are described in further detail in U.S. Provisional patent application Ser. No. 12/428,226, filed Apr. 22, 2009 (hereinafter “the '226 application”), which is hereby incorporated by reference in its entirety.

Further, the insertion tool 80 may comprise one or more markers disposed near the distal tip 82 and configured to be visualized under fluoroscopy of other imaging techniques to facilitate location of the distal tip 82. In the event the insertion tool is intended to penetrate tissue, the markers may help a physician determine how far the insertion tool 80 has penetrated into the tissue. Optionally, if the insertion tool comprises a needle-like body having a sharpened distal tip 82, a sheath member having an inner diameter larger than an outer diameter of the insertion tool 80 may be longitudinally advanced over the insertion tool 80, e.g., to cover the sharpened tip and optionally provide a blunt tip for pushing against tissue, as explained further in the '226 application.

In an alternative embodiment, the insertion tool 80 may comprise a catheter-like body having a substantially blunt distal tip. If the insertion tool 80 comprises a catheter-like body having a blunt distal tip, the end regions 49 of the distal deployable members 45-47 may be sharpened to penetrate tissue in lieu of penetration by the insertion tool 80.

Referring still to FIGS. 2-3, the hollow lumen 85 of the insertion tool 80 may comprise an inner diameter that is larger than an outer diameter of the tacking device 20. Therefore, the tacking device 20 may be loaded into the hollow lumen 54 in a delivery configuration, whereby the distal deployable members 45-47 are in the contracted state, as shown in FIGS. 2-3.

The insertion tool 80 may comprise a longitudinal slit 87 formed therein, preferably beginning at the distal tip 82 and extending in a proximal direction. The slit 87 may terminate at a location proximal to where the loop member 70 is positioned during delivery of the tacking device 20, as depicted in FIGS. 2-3. The slit 87 preferably is dimensioned to receive at least a portion of the loop member 70 and the suture 90 that is disposed through the loop member 70. In use, when the tacking device 20 is advanced longitudinally with respect to the insertion tool 80, the loop member 70 may move longitudinally within the slit 87.

As noted above, the first retainer 50 may be used in conjunction with a second retainer 60 that may be manipulated by a physician. The second retainer 60 may formed integral with or coupled to an actuating wire 62, which extends proximally and may be manipulated by a physician. In use, the first retainer 50 is configured to be distally advanced, via the actuating wire 62 and the second retainer 60, to at least partially deploy the tacking device 20 at a preliminary location. If it becomes desirable to reposition the partially or fully deployed tacking device 20, the actuating wire 62 and the second retainer 60 then may be proximally retracted, thereby proximally retracting the engaged first retainer 50 and associated tacking device 20, and allowing for repositioning of the distal deployable members 45-47 at a subsequent location. When a desired final location is achieved, the first retainer 50 is configured to be disengaged from the second retainer 60, leaving only the tacking device 20 inside the body, as explained in FIG. 9 below.

Various types of complementary first and second retainers 50 and 60 may be used to facilitate controlled release of the tacking device 20 in accordance with the present embodiments. Suitable complementary first and second retainers 50 and 60 are described in commonly-assigned U.S. patent application Ser. No. 11/807,827, filed May 30, 2007 (hereinafter “the '827 application”), which is hereby incorporated by reference in its entirety. The first and second retainers 50 and 60 shown in the present application therefore are one of multiple possible types of retaining mechanisms for controlled release of the tacking device 20.

The second retainer 60 is complementary to the first retainer 50 so that the first and second retainers 50 and 60 can be matingly joined. Accordingly, the first retainer 50 has a knob 55 disposed proximal to a notch 56, as shown in FIGS. 1-2. In a symmetrical manner, the second retainer 60 has a knob 65 disposed distal to a notch 66, as shown in FIG. 2. The knobs 55 and 65 may approximate the shape of a half-cylinder having a flat surface, as depicted in FIGS. 2-3, or alternatively may comprises a rounded configuration, as described further in the '827 application.

The first and second retainers 50 and 60 are joined with each other by locating the knob 55 of first retainer 50 within the notch 66 of the second retainer 60, and by locating the knob 65 of the second retainer 60 within the notch 56 of the first retainer 50. When joined, the first and second retainers 50 and 60 form a substantially continuous cylinder shape having substantially the same outer diameter, as shown in FIGS. 2-3. The outer diameter of the first and second retainers 50 and 60, when mated, preferably is slightly less than an inner diameter of the insertion tool 80, and further preferably is substantially identical to the outer diameter of the main body 22, as depicted in FIGS. 2-3.

It should be noted that although first retainer 50 matingly joins with second retainer 60, they will not retain a joined position unless they are held together. Since the insertion tool 80 comprises an inner diameter that is slightly larger than the mated first and second retainers 50 and 60, the insertion tool 80 therefore holds and maintains the first and second retainers 50 and 60 in a mating position, as long as the insertion tool 80 covers both the mating first and second retainers 50 and 60, as shown in FIGS. 2-3.

The tacking device 20 may be loaded into the insertion tool 80 outside of the patient's body. In a first step, the actuating wire 62 may be loaded into the insertion tool 80 such that the second retainer 60 extends just distal to the distal tip 82 of the insertion tool 80. The tacking device 20 is provided and the first retainer 50 is matingly joined with the second retainer 60, as described above. The mating first and second retainers 50 and 60 then are loaded through the distal tip 82 of the insertion tool 80 and advanced in a proximal direction. The remainder of the tacking device 20 then is advanced proximally into the insertion tool 80, with the loop member 70 being aligned with the slit 87, until the distal deployable members 45-47 are contracted as shown in FIGS. 2-3. In this state, the insertion tool 80 may be introduced into a body cavity via a working lumen of an insertion device, such as an endoscope, as explained further below.

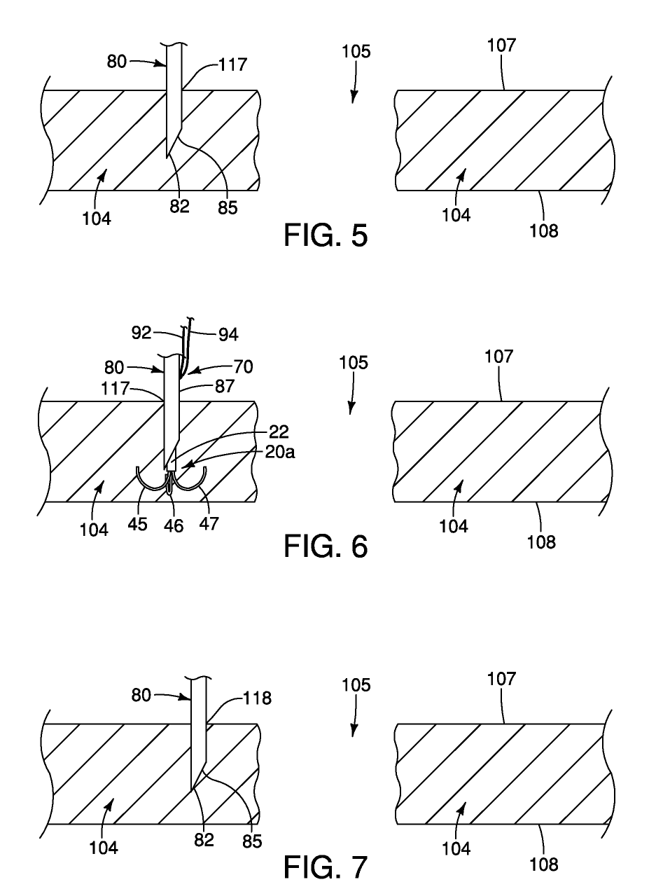

Referring now to FIG. 4, an insertion device 110 suitable for delivering the insertion tool 80 and one or more tacking devices 20 is shown. The insertion device 110 may comprise an endoscope having proximal and distal ends, and a working lumen 115 disposed therebetween. For illustrative purposes, the optical components of the endoscope are not shown in the side-sectional view of FIG. 4. However, if an endoscope is employed, it will be understood that the endoscope may comprise optical components, such as fiber optic elements, to facilitate visualization of objects distal to, or to the side of, the endoscope.

As shown in FIG. 4, the working lumen 115 of the insertion device 110 comprises an inner diameter that is larger than an outer diameter of the insertion tool 80, thereby permitting proximal and distal advancement of the insertion tool 80 with respect to the insertion device 110. As depicted, the loop member 70 may extend through the slit 87 of the insertion tool 80 and into the working lumen 115 of the insertion device 110. The suture 90 is looped through the loop member 70, such that the first and second ends 92 and 94 of the suture 90 extend through the working lumen 115 adjacent to the insertion tool 80, as depicted in FIG. 4.

Referring now to FIGS. 5-10, one exemplary method of use of the tacking device 20 and the insertion tool 80 is described. In FIGS. 5-10, multiple tacking devices are used to facilitate closure of an opening 105 in tissue 104. The tissue 104 comprises a mucosal layer 107 and a serosal layer 108. By way of example, the opening 105 may be formed during a translumenal procedure, whereby the tissue 104 may comprise tissue of the stomach, small or large intestines, or another bodily passage.

In order to facilitate closure of the opening 105, a plurality of tacking devices 20 are disposed at least partially through the tissue 104 at one or more locations in the vicinity of the opening 105. Preferably, multiple tacking devices 20 at least partially surround the perimeter of the opening 105. In the embodiment of FIGS. 5-10, a first tacking device 20a is disposed on one side of the opening 105, and a second tacking device 20b is disposed on a substantially opposing side of the opening 105.

In a first step, the insertion device 110 of FIG. 4 may be advanced through a bodily lumen such as the alimentary canal to a position proximate the target tissue 104. Preferably, this is accomplished with the assistance of an endoscope or laparoscope, although the tacking device could also be used in open surgery. The insertion tool 80, with the first tacking device 20a loaded therein in the contacted state, is positioned within the working lumen 115 of the insertion device 110. At this time, the second retainer 60 is coupled to the first retainer 50, as explained in FIGS. 2-4 above. Further, the suture 90 is coupled to the first tacking device 20a by being looped through the loop member 70, which extends through the slit 87 in the insertion tool 80, as shown in FIG. 4.

Under visualization using the insertion device 110 and/or another suitable imaging modality, the insertion tool 80 may be advanced in a distal direction with respect to the insertion device 110. The insertion tool 80 may pierce partially through the tissue 104 at a preliminary location 117 around the perimeter of the opening 105, as shown in FIG. 5. The sharpened distal tip 82 of the insertion tool 80 may be advanced to a predetermined depth into the tissue 104, while the first tacking device 20a is in the contracted delivery state within the hollow lumen 85. As noted above, markers on the insertion tool 80 may facilitate in determining how far the insertion tool 80 has penetrated into the tissue 104.

Referring to FIG. 6, in a next step the physician may at least partially deploy the first tacking device 20a at the preliminary location 117. In one technique, the actuating wire 62 and associated second retainer 60 may be advanced in a distal direction with respect to the insertion tool 80. Since the second retainer 60 is still coupled to the first retainer 50, the distal movement of the actuating wire 62 correspondingly moved the first tacking device 20a in a distal direction with respect to the insertion tool 80. As shown in FIG. 6, when the distal deployable members 45-47 are no longer radially constrained by the insertion tool 80, they may assume their predetermined expanded configurations in which they may engage, penetrate and/or abut the tissue 104. It should be noted that the loop member 70 may slide distally within the slit 87 of the insertion tool 80 as the first tacking device 20 is distally advanced. Further, it should be noted that in an alternate deployment technique, the actuating wire 62 and associated second retainer 60 may be held steady, and the insertion tool 80 may be proximally retracted with respect to the first tacking device 20a to at least partially deploy the distal deployable members 45-47.

In accordance with one aspect, the physician may determine that the first tacking device 20a is desired to be repositioned. For example, the physician may realize that the main body 22 and/or the distal deployable members 45-47 have been deployed too close, or too far, from the opening 105 in the tissue 104, or that the distal deployable members 45-47 have been deployed in an undesirable orientation. Since the first retainer 50 is still engaged with the second retainer 60 within the insertion tool 80, as described in FIGS. 2-4, the physician may “recapture” and subsequently reposition the first tacking device 20a. In particular, the physician may proximally retract the actuating wire 62, which retracts the second retainer 60, the first retainer 50 coupled thereto, and the associated first tacking device 20a. When the first tacking device 20a is proximally retracted with respect to the insertion tool 80, the distal deployable members 45-47 may be brought back into the lumen 85 of the insertion tool 80 and assume the contracted state, shown in FIGS. 2-4. In an alternate technique, the physician may distally advance the insertion tool 80, while the actuating wire 62 is held steady, to advance the insertion tool 80 over the distal deployable members 45-47. When the distal deployable members 45-47 are within the confines of the hollow lumen 85, the physician then may proximally retract the insertion tool 80 and first tacking device 20a, in tandem, so that the insertion tool 80 and the first tacking device 20a are no longer engaged with the tissue 104.

Preferably, the physician recaptures the distal deployable members 45-47 in the same manner in which they were deployed, i.e., either the insertion tool 80 is advanced and retracted while the retainers are always held steady, or vice versa. By deploying and recapturing in the same manner, the tacking device may enter and exit the tissue in substantially the same path, which may reduce trauma to the tissue. It should also be noted that if a sheath member is employed and configured to be longitudinally advanced over the insertion tool 80, e.g., to cover the sharpened tip, the sheath member may also be used to recapture the tacking device by being distally advanced relative to both the insertion tool 80 and the partially deployed tacking device. Preferably, the distal deployable members 45-47 comprise substantially smooth outer surfaces to prevent snagging on the tissue 74 when moved from the expanded to contracted states, and more preferably, the distal deployable members 45-47 are free of barb-like elements.

Referring now to FIGS. 7-8, in a next step, the physician may reposition the insertion tool 80 at another location within the tissue 104, such as a first final location 118. The insertion tool 80 may pierce the tissue 104 at the first final location 118, as shown in FIG. 7, and the first tacking device 20a may be distally advanced with respect to the insertion tool 80 to cause the distal deployable members 45-47 to engage the tissue 104, as shown in FIG. 8 and described above.

Referring now to FIG. 9, if the deployment of the first tacking device 20a is deemed acceptable, the physician then may release the first tacking device 20a. Specifically, the insertion tool 80 may be retracted proximally with respect to the first tacking device 20a, while the actuating wire 62 is held steady, to expose the junction between the first and second retainers 50 and 60. When the insertion tool 80 passes the first and second retainers 50 and 60, they detach and release from each other. The first tacking device 20a is left inside the body, with the distal deployable members 45-47 engaging the tissue 104. It should be noted that the loop member 70 of the first tacking device 20 preferably is disposed proximal to the mucosal layer 107 of the tissue 104, as shown in FIG. 9. The suture 90 remains looped through the loop member 70, with the first and second ends 92 and 94 of the suture 90 extending through the working lumen 115 of the insertion device 110 and outside of the body.

In a next step, the insertion tool 80 may be proximally retracted until the distal tip 82 is outside of the insertion device 110 and the patient's body. A second tacking device 20b then may be loaded into the insertion tool 80, preferably in the same manner described above. In particular, the first and second retainers 50 and 60 are matingly joined, then the tacking device 20b is loaded into the insertion tool 80 in a proximal to distal direction until the distal deployable members 45-47 are contracted as shown in FIGS. 2-3. At this time, the loop member 70 of the second tacking device 20b is aligned with the slit 87 of the insertion tool 80. One free end of the suture 90 then may be looped through the loop member 70 of the second tacking device 20b. The insertion tool 80 then may be advanced through the working lumen 115 of the insertion device 110, while the suture 90 is disposed partially within the working lumen 115 with the first and second ends 92 and 94 of the suture 90 configured to be manipulated by a physician.

Referring to FIG. 10, the second tacking device 20b may be deployed at a second final location 119 in the tissue 104 in the manner described above for the first tacking device 20b. Notably, the physician may recapture and reposition the tacking device 20b after the distal deployable members 45-47 have been at least partially deployed, so long as the first and second retainers 50 and 60 remain joined. Once the final positioning is acceptable, the physician may proximally retract the insertion tool 80 with respect to the tacking device 20b to expose the junction between the first and second retainers 50 and 60, thereby detaching the retainers and leaving the second tacking device 20b at the second final location 119. It should be noted that the suture 90 remains looped through both the loop members 70 of the first and second tacking device 20a and 20b, as shown in FIG. 10, with the free ends 92 and 94 extending outside of the body for manipulation by a physician. In this manner, any number of subsequent tacking device 20 may be inserted and deployed to at least partially surround the perimeter of the opening 105.

The two free ends 92 and 94 of the suture 90 may be independently tensioned to facilitate closure of the opening 105. When the ends 92 and 94 are tensioned, it reduces the distance between the tacking devices and compresses the tissue 104 around the perforation 105. Preferably, multiple tacking devices having loop members 70 are sequentially positioned to at least partially surround the perforation 105 in a semi-annular or annular shape. The suture ends 92 and 94 may be secured to maintain the compression of the tissue 104 using any suitable technique such as by forming a knot or using clamps, rivets and the like.

Further, in lieu of the loop members 70 described herein, other mechanisms for engaging and/or retaining sutures may be integrally formed with the tacking device 20 or externally attached thereto. Solely by way of example, such suture retaining mechanisms are explained in pending U.S. patent application Ser. No. 11/946,565, filed Nov. 28, 2007, the entire disclosure of which is hereby incorporated by reference in its entirety.

Various types of sutures 90 may be used in conjunction with the embodiments herein. For example, synthetic sutures may be made from polypropylene, nylon, polyamide, polyethylene, and polyesters such as polyethylene terephthalate. These materials may be used as monofilament suture strands, or as multifilament strands in a braided, twisted or other multifilament construction. However, the tacking device 20 also could be used to tack down a graft or other implantable member, without the use of a suture system.

While the examples shown above have illustratively described tacking devices that may be useful for closing openings in bodily walls during translumenal procedures, the tacking devices 20 also may be used in other procedures. For example, the tacking devices 20 may be used for coupling a graft member to tissue to cover and seal a perforation, such as a hernia, and may be used to secure a graft member to tissue for reconstructing local tissue, or for treating anatomoses, and the like.

Further, the tacking device 20 may be used to grasp or manipulate tissue 104, and then be withdrawn from the body. For example, after the distal deployable members 45-47 have been deployed into the tissue 104 as shown in FIG. 6 above, a physician may manipulate the tissue 104 via the engaged distal deployable members 45-47, e.g., in a manner similar to a forceps, except that the manipulation occurs from within the tissue 104 as opposed to externally from the mucosal layer 107 only. After the desired manipulation has occurred, the distal deployable members 45-47 may be removed from engagement with the tissue 104, as set forth above, and subsequently permanently deployed or removed from the body.

In further alternative embodiments, the apparatus and methods described herein may be used for engaging a layer of material, and are not restricted to methods for treatment of a human or animal body by surgery or therapy. For example, the first tacking device may be inserted to a position proximate a layer of material with the distal deployable members in the contracted states. The distal deployable members may be at least partially expanded to engage the layer of material at a preliminary location. A first retainer disposed at the proximal end of the main body may be proximally retracted to thereby proximally retract and contract the distal deployable members. The first tacking device may be repositioned at a first final location, and the distal deployable members may be deployed to engage the layer of material in the expanded state at the first final location, as generally described above.

While various embodiments of the invention have been described, the invention is not to be restricted except in light of the attached claims and their equivalents. Moreover, the advantages described herein are not necessarily the only advantages of the invention and it is not necessarily expected that every embodiment of the invention will achieve all of the advantages described.

I claim:

1. A tacking device for engaging tissue, the tacking device comprising:
a main body having proximal and distal ends;
at least one distal deployable member having contracted and expanded states, wherein the distal deployable members extend distally from the distal end of the main body and are configured to engage tissue in the expanded state;
an insertion tool comprising a hollow lumen, wherein the tacking device is adapted to be disposed within the hollow lumen during delivery with the distal deployable members in the contracted states;
a first retainer disposed at the proximal end of the main body; and
a second retainer configured to be coupled to the first retainer when the insertion tool is positioned over both the first and second retainers, and further configured to be disengaged from the first retainer when no longer covered by the insertion tool, and
wherein, after the distal deployable members have been at least partially expanded at a preliminary location, the distal deployable members are configured to be contracted within the hollow lumen of the insertion tool and repositioned at a final location.

2. The tacking device of claim 1 wherein, after the distal deployable members have been at least partially expanded at the preliminary location, proximal retraction of an actuating wire coupled to the second retainer is adapted to cause a corresponding proximal retraction of the first retainer to proximally retract the distal deployable members within the insertion tool, thereby assuming the contracted state and facilitating repositioning of the distal deployable members at the final location.

3. The tacking device of claim 1 wherein, after the distal deployable members have been at least partially expanded at the preliminary location, distal advancement of the insertion tool relative to the tacking device, while the first and second retainers are held steady and coupled to one another, is operative to contract the distal deployable members within the hollow lumen of the insertion tool and permit repositioning at the final location.

4. The tacking device of claim 1 wherein the first retainer comprises a notch and further comprises a knob formed proximal to the notch, and wherein the second retainer comprises a notch and further comprises a knob disposed distal to the notch, wherein the knob of the second retainer is aligned with the notch of the first retainer, and wherein the knob of the first retainer is aligned with the notch of the second retainer to secure the first retainer to the second retainer.

5. The tacking device of claim 1 further comprising a loop member having an aperture disposed near the proximal end of the main body, wherein the aperture is sized to receive a suture.

6. The tacking device of claim 5, wherein the loop member is positioned proximal to a mucosal layer of the tissue when the distal deployable members engage the tissue in the expanded state.

7. The tacking device of claim 1, wherein the distal deployable members comprise hook-shaped configurations in the expanded states in which the distal deployable members retroflex about 180 degrees relative to the main body.

8. The tacking device of claim 7, wherein the distal deployable members are constructed from a nickel-titanium alloy that is configured to self-expand to the hook-shaped configurations.

9. The tacking device of claim 1, wherein the distal deployable members are configured to move in a radial and proximal direction relative to the main body when expanding from the contracted state to the expanded state.

10. The tacking device of claim 1 wherein the distal deployable members comprise substantially smooth outer surfaces configured to prevent snagging on tissue when moved from the expanded to contracted states.

11. Apparatus for facilitating closure of a bodily opening, the apparatus comprising:
a first tacking device comprising a main body having proximal and distal ends, and further having at least one distal deployable member having contracted and expanded states, wherein the distal deployable members extend distally from the distal end of the main body and are configured to engage tissue at a first final location in the vicinity of an opening in the tissue;
a second tacking device comprising a main body having proximal and distal ends, and further having at least one distal deployable member having contracted and expanded states, wherein the distal deployable members extend distally from the distal end of the main body and are configured to engage tissue at a second final location in the vicinity of the opening in the tissue;
a first loop member comprising an aperture disposed near the proximal end of the main body of the first tacking device;
a second loop member comprising an aperture disposed near the proximal end of the main body of the second tacking device; and
a suture configured to be slidably disposed through the apertures of the first and second loop members and actuated in a purse-string fashion to facilitate closure of the opening in the tissue when the first and second tacking devices are deployed at the first and second final locations,
wherein the distal deployable members of the first and second tacking devices comprise substantially smooth outer surfaces configured to prevent snagging on tissue when moved between the contracted and expanded states.

12. The apparatus of claim 11 wherein, after the distal deployable members of the first tacking device have been at least partially expanded at a preliminary location, the distal deployable members of the first tacking device are configured to be contracted within a hollow lumen of an insertion device and repositioned at the first final location.

13. The apparatus of claim 11, wherein the distal deployable members of the first and second tacking devices comprise hook-shaped configurations in the expanded states in which the distal deployable members retroflex about 180 degrees relative to the main body.

14. The apparatus of claim 11 further comprising:
an insertion tool comprising a hollow lumen, wherein the first tacking device is adapted to be disposed within the hollow lumen during delivery with the distal deployable members in the contracted states; and
a slit formed in a distal region of the insertion tool, wherein the first loop member is adapted to extend at least partially through the slit and is configured for longitudinal movement within the slit, and
wherein the suture is configured to extend outside of the insertion tool during delivery of the first tacking device.

15. A method for controlled release of a tacking device into tissue, the method comprising:
providing a first tacking device comprising a main body having proximal and distal ends, and further comprising at least one distal deployable member having contracted and expanded states that extends distally from the distal end of the main body;
inserting the first tacking device to a position proximate the tissue with the distal deployable members in the contracted states;
at least partially expanding the distal deployable members of the first tacking device to engage tissue at a preliminary location;
proximally retracting a first retainer disposed at the proximal end of the main body to thereby proximally retract and contract the distal deployable members;
repositioning the first tacking device at a first final location; and
deploying the distal deployable members to engage tissue in the expanded state at the first final location.

16. The method of claim 15 further comprising:
providing a loop member comprising an aperture disposed near the proximal end of the main body of the first tacking device;
disposing a suture through the aperture of the loop member of the first tacking device; and
manipulating the suture to thereby manipulate at least a portion of the first tacking device.

17. The method of claim 16 further comprising:
providing a second tacking comprising a main body having proximal and distal ends, at least one distal deployable member extending distally from the distal end of the main body, and further comprising a loop member comprising an aperture disposed near the proximal end of the main body of the second tacking device;
disposing the suture through the aperture of the loop member of the second tacking device;
deploying the distal deployable members of the second tacking device to engage tissue in the expanded state at a second final location, wherein the first and second final locations at least partially surround an opening in the tissue; and
actuating ends of the suture in a purse-string fashion to facilitate closure of the opening in the tissue.

18. The method of claim 17, wherein the loop member of the first tacking device and the loop member of the second tacking device are disposed proximal to a mucosal surface of the tissue when the distal deployable members are in the expanded states.

19. The method of claim 15 further comprising:
coupling the first retainer to a second retainer prior to deployment of the first tacking device; and
disengaging the first retainer from the second retainer after the first tacking device is deployed at the first final location.

20. The method of claim 19 further comprising:
providing an insertion tool comprising a hollow lumen, wherein the first tacking device is disposed within the hollow lumen during delivery with the distal deployable members in the contracted states;
coupling the first and second retainers together when the insertion tool is positioned over both the first and second retainers; and
disengaging the first retainer from the second retainer when a junction of the first and second retainers is no longer covered by the insertion tool.
